(19) United States
(12) Patent Application Publication
(10) Pub. No.: US 2024/0025947 A1
(43) Pub. Date: Jan. 25, 2024

(54) PREPARATION AND PURIFICATION PROCESS OF MONOMETHYL AURISTAIN E COMPOUND

(71) Applicant: REMEGEN CO., LTD., Yantai (CN)

(72) Inventors: Zhuanglin LI, Yantai (CN); Wei GUO, Yantai (CN); Peng SUN, Yantai (CN); Kai XIAO, Yantai (CN); Xinli LI, Yantai (CN)

(21) Appl. No.: 18/374,902

(22) Filed: Sep. 29, 2023

Related U.S. Application Data
(63) Continuation of application No. PCT/CN2022/084236, filed on Mar. 31, 2022.

(30) Foreign Application Priority Data
Mar. 31, 2021 (CN) 202110349993.3

(51) Int. Cl. C07K 7/02; C07K 1/34; C07K 1/12

(52) U.S. Cl. CPC C07K 7/02; C07K 1/34; C07K 1/12

(57) ABSTRACT

Provided is a preparation and purification process of MMAE. The process has mild synthesis and purification conditions, can effectively prevent the change of product chirality caused by excessively high temperature, greatly reduces generation of degradation impurities, and increases the purity and yield of the product. In addition, the preparation and purification process has good stability and is more suitable for scale-up production. The MMAE prepared has purity of higher than 99%, and can perfectly meet clinical drug requirements.

### FIELD

The invention relates to the technical field of compound synthesis, in particular to a preparation and purification process of a monomethyl auristatin E compound (ie. MMAE).

### BACKGROUND

MMAE (Monomethyl Auristantin E, also known as methyl auristatin E), a fully synthetic derivative of auristatin, can effectively inhibit mitosis by inhibiting tubulin polymerization, and has been widely used as a cytotoxic component (ie, the drug moiety) to synthesize antibody-drug conjugates to treat cancer.

Antibody drug conjugate (ADC) is a class of antitumor drugs, and includes three components: Antibody, Linker and Drug. Its principle is that the selective targeting ability of the antibody is combined with the cytotoxic efficacy of the drug moiety, then an antigen on the surface of a tumor cell is specifically recognized by means of the targeting specificity of the antibody, entry of the cell is achieved through endocytosis of the cell, the drug moiety is released by protease in the cell, and thus, the purposes of killing the tumor cell while avoiding killing of non-target tissues are achieved.

Currently, a large number of natural and chemically synthesized cytotoxins are known to exist, but only a very small part of the drug structure can be applied to ADCs. This is mainly because toxins that can be used as ADC loads must have complex properties such as high cytotoxic potency and small molecular weight. Therefore, auristatin compounds (such as MMAE) are highly sought after in the ADC field. Currently, the linker-toxin structure on antibodies of many ADC drugs on the market is Mc-Val-Cit-PAB-MMAE. However, the current market price of MMAE is very high. The main reason is that the current synthesis and purification process is still immature, the synthesis process of many drugs is complex, and the purification process is immature, which result in low yield, low purity and high content of impurities (especially the single impurity) of the final product. While for the safety of clinical medication, the drug used for clinical use must have extremely high purity and extremely low impurities, but most of the current processes cannot meet the standard requirements of clinical medication, which is also one of the main reasons for the high price of the commercially available MMAE.

### SUMMARY

The invention provides a preparation and purification process capable of obtaining extremely high-purity MMAE (structural formula as shown in formula I), which can well meet the quality requirements of clinical drugs.

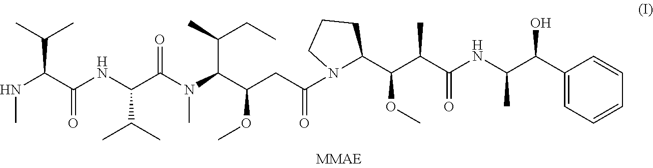 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

The preparation route of the method is as follows:

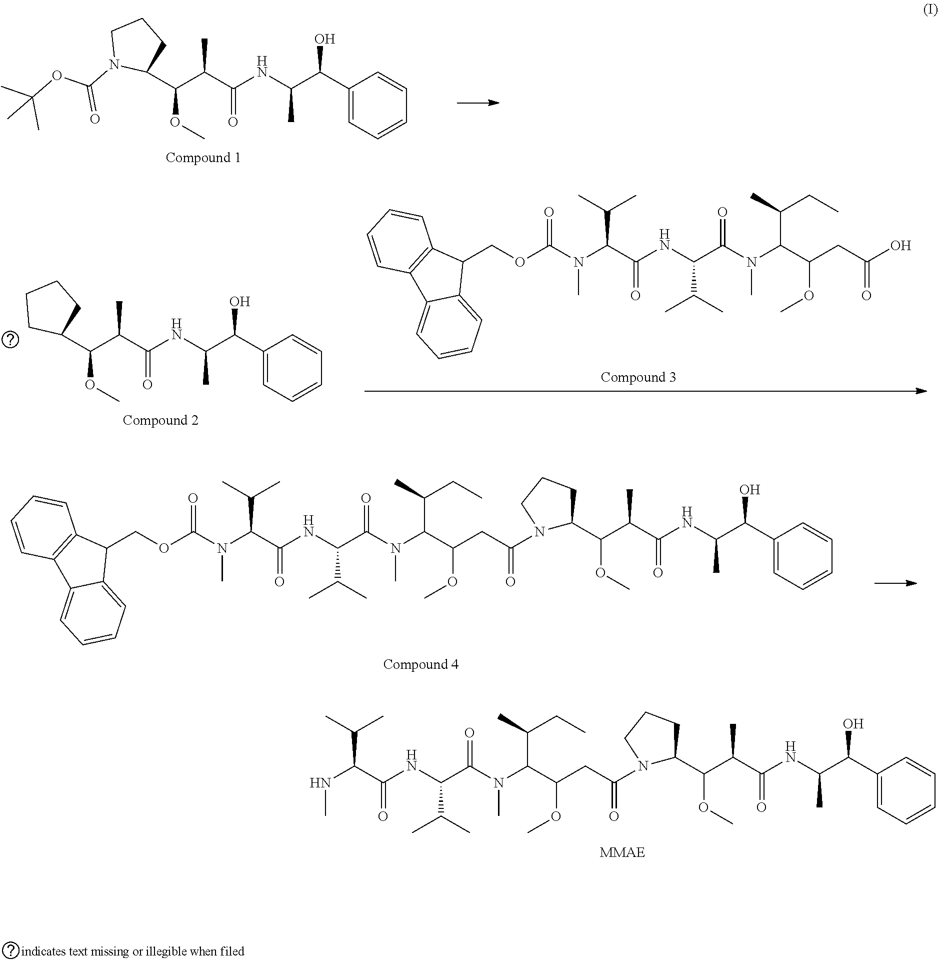

The method includes the following steps:
(1) dissolving a compound 1 in an appropriate amount of a first organic solvent to form a solution A;
(2) adding a sufficient amount of HCl-1,4-dioxane solution to the solution A for insulation reaction, and removing a Boc protecting group;
(3) after the reaction finishes, pouring the reaction solution of step (2) into a sufficient amount of first low-polarity solvent, discarding the filtrate after stirring, and solid residues being compound 2 after drying;
(4) dissolving the obtained compound 2 and an appropriate amount of compound 3 in a second organic solvent to form a solution B;
(5) dissolving a first polypeptide condensing agent in an appropriate amount of third organic solvent to form a solution C, where the mole number of the first polypeptide condensing agent is larger than the mole number of the compound 3 of step (4);
(6) adding the solution C to the solution B to form a solution D;
(7) adding an appropriate amount of first organic base to the solution I) for insulation reaction;
(8) after the reaction of step (7) finishes, adding a sufficient amount of second low polarity solvent and purified water to the reaction system of step (7) for extraction, and collecting an organic phase;
(9) washing the organic phase collected in step (8) with an appropriate amount of hydrochloric acid solution, purified water and sodium chloride solution successively, drying by anhydrous sodium sulfate, concentrating under reduced pressure, and drying to obtain a compound 4;
(10) dissolving the compound 4 in a fourth organic solvent to form a solution E;
(11) adding a sufficient amount of diethylamine to the solution E for insulation reaction, removing a Fmoc protecting group;
(12) after the reaction finishes, adding an appropriate amount of fifth organic solvent and purified water to the reaction system of step (11) for extraction, collecting an organic phase, drying by anhydrous sodium sulfate, and concentrating under reduced pressure;
(13) carrying out chromatographic purification on the concentrate under reduced pressure obtained in step (12) by an elution system of toluene:methanol, and concentrating the collected eluent under reduced pressure;
(14) after dissolving the concentrated product under reduced pressure obtained in step (13) with a sixth organic solvent, filtering, and concentrating the filtrate under reduced pressure; and
(15) vacuum-drying the concentrated product under reduced pressure obtained in step (14) to obtain the MMAE.

Further, the first organic solvent in step 1 is selected from dichloromethane, trichloromethane and carbon tetrachloride; and preferably, the first organic solvent in step 1 is dichloromethane.

Rather, the weight-to-volume ratio (g/mL) of the compound 1 to the first organic solvent in step 1 is about 1:2; preferably, the weight-to-volume ratio (g/mL) of the compound 1 to the first organic solvent in step 1 is 1:1-3; and more preferably, the weight-to-volume ratio (g/mL) of the compound 1 to the first organic solvent in step 1 is 1:1.5-2.5.

Further, the concentration of the HCl-1,4-dioxane solution in step (2) is about 4 mol/L; preferably, the concentration of the HCl-1,4-dioxane solution in step (2) is about 3-7 mol/L; and more preferably, the concentration of the HCl-1,4-dioxane solution in step (2) is 3.5-4.5 mol/L.

Further, the weight-to-volume ratio (g/mL) of the compound 1 in step 1 to the HCl-1,4-dioxane solution in step (2) is about 1:6; preferably, the weight-to-volume ratio (g/mL) of the compound 1 in step 1 to the HCl-1,4-dioxane solution in step (2) is 1:4-8; more preferably, the weight-to-volume ratio (g/mL) of the compound 1 in step 1 to the HCl-1,4-dioxane solution in step (2) is 1:5-7; and more preferably, the weight-to-volume ratio (g/mL) of the compound 1 in step 1 to the HCl-1,4-dioxane solution in step (2) is 1:6.

Further, the HCl-1,4-dioxane solution in step (2) is added dropwise, and the internal temperature of the reaction system is maintained between −5-5° C., during the dropwise addition.

Further, the temperature of the insulation reaction in step (2) is 10-15° C.

Further, the first low-polarity solvent in step (3) is selected from n-hexane, petroleum ether, and n-heptane; and preferably, the first low-polarity solvent is selected from n-hexane.

Further, the weight-to-volume ratio (g/mL) of the compound 1 in step 1 to the first low-polarity solvent in step (3) is about 1:16; preferably, the weight-to-volume ratio (g/mL) of the compound 1 in step 1 to the first low-polarity solvent in step (3) is 1:10-25; more preferably, the weight-to-volume ratio (g/mL) of the compound 1 in step 1 to the first low-polarity solvent in step (3) is 1:12-20; and more preferably, the weight-to-volume ratio (g/mL) of the compound 1 in step 1 to the first low-polarity solvent in step (3) is 1:15-17.

Further, the second organic solvent in step (4) is selected from DMF, DMA, DMSO and DCM; preferably, the second organic solvent is DMF; and further preferably, in step (4), the molar amounts of the compound 2 and the compound 3 are the same.

Further, the weight-to-volume ratio (g/mL) of the compound 3 to the second organic solvent in step (4) is about 1:6; preferably, the weight-to-volume ratio (g/mL) of the compound 3 to the second organic solvent in step (4) is 1:5-10; more preferably, the weight-to-volume ratio (g/mp) of the compound 3 to the second organic solvent in step (4) is 1:5-8; and even more preferably, the weight-to-volume ratio (g/mL) of the compound 3 to the second organic solvent in step (4) is 1:6-7.

Further, the first polypeptide condensing agent in step (5) is selected from HATU, DIC, DCC, EDC, HCTU, DEPBT, EEDQ and CDI; and preferably, the first polypeptide condensing agent in step (5) is HATU.

Further, the molar ratio of the compound 3 in step (4) to the first polypeptide condensing agent in step (5) is about 1:1.2; preferably, the molar ratio of the compound 3 in step (4) to the first polypeptide condensing agent in step (5) is 1:1.01-1.5; more preferably, the molar ratio of the compound 3 in step (4) to the first polypeptide condensing agent in step (5) is 1:1.1-1.4; and even more preferably, the molar ratio of the compound 3 in step (4) to the first polypeptide condensing agent in step (5) is 1:1.2-1.3.

Further, the third organic solvent in step (5) is selected from DMF, DMA, DMSO, and DCM; and preferably, the third organic solvent is DMF.

Further, the weight-to-volume ratio (g/mL) of the compound 3 in step (4) to the third organic solvent in step (5) is about 1:3; preferably, the weight-to-volume ratio (g/mL) of the compound 3 in step (4) to the third organic solvent in step (5) is 1:2-6; more preferably, the weight-to-volume ratio (g/mL) of the compound 3 in step (4) to the third organic solvent in step (5) is 1:2.5-4; and more preferably, the weight-to-volume ratio (g/mL) of the compound 3 in step (4) to the third organic solvent in step (5) is 1:3-4.

Further, in step (6), the solution C is added dropwise to the solution B, and the internal temperature of the entire reaction system during the dropwise addition is 0-5° C.

Further, the first organic base in step (7) is one or more selected from N,N diisopropylethylamine, triethylamine, and pyridine; and preferably, the first organic base in step (7) is N,N-diisopropylethylamine.

Further, the molar ratio of the compound 3 in step (4) to the first organic base in step (7) is about 1:3; preferably, the molar ratio of the compound 3 in step (4) to the first organic base in step (7) is 12-5; more preferably, the molar ratio of the compound 3 in step (4) to the first organic base in step (7) is 1:2.5-4; and even more preferably, the molar ratio of the compound 3 in step (4) to the first organic base in step (7) is 1:3-4.

Further, the first organic base in step (7) is added dropwise to the solution D, and the temperature of the insulation reaction is 0-5° C.

Further, the second low-polarity solvent in step (8) is selected from methyl tert-butyl ether, ethyl acetate, dichloromethane, and tetrahydrofuran; and preferably, the second low-polarity solvent in step (8) is methyl tert-butyl ether.

Further, the weight-to-volume ratio (g/mL) of the compound 3 in step (4) to the second low-polarity solvent and purified water in step (8) is about 1:20.2:20 preferably, the weight-to-volume ratio (g/mL) of the compound 3 in step (4) to the second low-polarity solvent and purified water in step (8) is 1:15-25:15-25; more preferably, the weight-to-volume ratio (g/mL) of the compound 3 in step (4) to the second low-polarity solvent and purified water in step (8) is 1:20-24:20-24; and more preferably, the weight-to-volume ratio (g/mL) of the compound 3 in step (4) to the second low-polarity solvent and purified water in step (8) is 1:20-21:20-21.

Further, the volumes of the second low-polarity solvent and the purified water in step (8) are the same.

Further, the concentration of the hydrochloric acid solution in step (9) is about 0.05 mol/L preferably, the concentration of the hydrochloric acid solution in step (9) is 0:02-0.08 mol/L; more preferably, the concentration of the hydrochloric acid solution in step (9) is 0.04-0.06 mol/L; and more preferably, the concentration of the hydrochloric acid solution in step (9) is 0.05 mol/L.

Further, the concentration of the sodium chloride solution in step (9) is about 30%; and preferably, the concentration of the sodium chloride solution in step (9) is 20%-40%.

Further, the volume of the hydrochloric acid solution, the purified water and the sodium chloride solution in step (9) is equal to the volume of the second low-polarity solvent in step (8).

Further, the fourth organic solvent in step (10) is selected from dichloromethane, acetonitrile, trichloromethane and carbon tetrachloride; preferably, the fourth organic solvent in step (10) is dichloromethane.

Further, the weight-to-volume ratio (g/mL) of the compound 4 to the fourth organic solvent in step (10) is about 1:7; preferably, the weight-to-volume ratio (g/mL) of the compound 4 to the fourth organic solvent in step (10) is 1:4-10; and more preferably, the weight-to-volume ratio (g/mL) of the compound 4 to the fourth organic solvent in step (10) is 1:5-8.

Further, wherein the weight-to-volume ratio (g/mL) of the compound 4 in step (10) to the diethylamine in step (11) is about 1:3.5; preferably, the weight-to-volume ratio (g/mL) of the compound 4 in step (10) to the diethylamine in step (11) is 1:3-5; and more preferably, the weight-to-volume ratio (g/mL) of the compound 4 in step (10) to the diethylamine in step (11) is 1:3-4.

Further, the diethylamine in step (11) is added dropwise to the solution E, and in the dropwise addition process, the internal temperature of the solution is kept between 0° C. and and the temperature of the insulation reaction in step (11) is 20-30° C.

Further, the fifth organic solvent in step (12) is selected from dichloromethane, trichloromethane, carbon tetrachloride and toluene; and preferably, the fifth organic solvent in step (12) is dichloromethane.

Further, the weight-to-volume ratio (g/mL) of the compound 4 in step (10) to the fifth organic solvent and purified water in step (12) is about 1:7:10; preferably, the weight-to-volume ratio (g/mL) of the compound 4 in step (10) to the fifth organic solvent and purified water in step (12) is 1:5-10:5-15, and more preferably, the weight-to-volume ratio (g/ml) of the compound 4 in step (10) to the fifth organic solvent and purified water in step (12) is 1:6-8:9-12.

Further, the silica gel used in the chromatographic purification in step (13) is 200-300 mesh silica gel; the elution system is toluene:methanol in a volume ratio (V/V) of 10-20:1; preferably, the elution system is firstly toluene:methanol in a volume ratio (V/V) of about 20:1. When TLC detects that only the product is visible (for example, only product spots), the elution system is changed to toluene:methanol in a volume ratio (V/V) of about 10:1. Of course, the elution system may not be replaced, and the purpose of replacing the elution system here is to make the product elated more quickly and to save time and cost in the production process.

Further, the developing agent of the TLC detection is toluene:methanol in a volume ratio (V/V) of about 5:1.

Further, the sixth organic solvent in step (14) is selected from methanol, toluene and acetonitrile; and preferably, the sixth organic solvent in step (14) is methanol.

Further, the weight-to-volume ratio (g/mL) of the compound 4 in step (10) to the sixth organic solvent in step (14) is about 1:3-10.

Further, the process in step (14) may be repeated 1-5 times.

The invention also provides a preparation and purification method of a compound shown in the following formula:

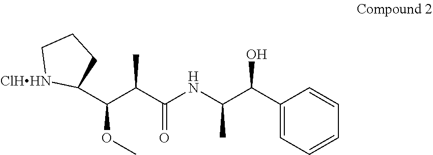

The preparation route of the method is as follows:

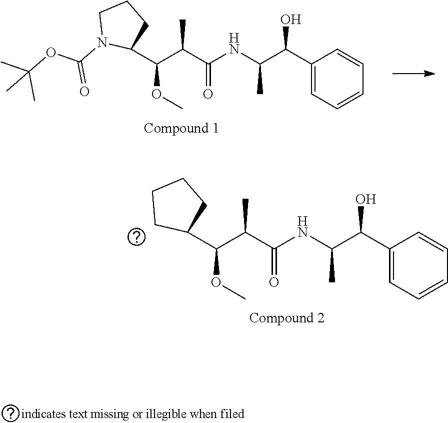

The method includes the following steps:
(1-1). dissolving a compound 1 in an appropriate amount of seventh organic solvent to form a solution F;
(1-2). adding a sufficient amount of HCl-1,4-dioxane solution to the solution F for insulation reaction, and removing a Boc protecting group; and
(1-3). after the reaction finishes, pouring the reaction solution of step (1-2) into a sufficient amount of third low-polarity solvent, discarding a filtrate after stirring, and solid residues being a compound 2 after drying.

Further, the seventh organic solvent in step (1-1) is selected from dichloromethane, trichloromethane and carbon tetrachloride; and preferably, the first organic solvent in step (1-1) is dichloromethane.

Further, the weight-to-volume ratio (g/mL) of the compound 1 to the seventh organic solvent in step (1-1) is about 1:2; preferably, the weight-to-volume ratio (g/mL) of the compound 1 to the seventh organic solvent in step (1-1) is 1:1-3; and more preferably, the weight-to-volume ratio (g/mL) of the compound 1 to the seventh organic solvent in step (1-1) is 1:1.5-2.5.

Further, the concentration of the HCl-1,4-dioxane solution in step (1-2) is about 4 mol/L; preferably, the concentration of the HCl-1,4-dioxane solution in step (1-2) is about 3-7 mol/L; and more preferably, the concentration of the HCl-1,4-dioxane solution in step (1-2) is 3.5-4.5 mol/L.

Further, the weight-to-volume ratio (g/mL) of the compound 1 in step (1-1) to the HCl-1,4-dioxane solution in step (1-2) is about 1:6; preferably, the weight-to-volume ratio (g/mL) of the compound 1 in step (1-1) to the HCl-1,4-dioxane solution in step (1-2) is 1:4-8; more preferably, the weight-to-volume ratio (g/mL) of the compound 1 in step (1-1) to the HCl-1,4-dioxane solution in step (1-2) is 1:5-7; and even more preferably, the weight-to-volume ratio (g/mL) of the compound 1 in step (1-1) to the HCl-1,4-dioxane solution in step (1-2) is 1:6.

Further, the HCl-1,4-dioxane solution in step (1-2) is added dropwise, and the internal temperature of the reaction system is maintained between −5-5° C. during the dropwise addition.

Further, the temperature of the insulation reaction in step (1-2) is 10-15° C.

Further, the third low-polarity solvent in step (1-3) is selected from n-hexane, petroleum ether, and n-heptane; and preferably, the third low-polarity solvent in step (1-3) is selected from n-hexane.

Further, the compound 1 in step (1-1) to the third low-polarity solvent in step (1-3) is about 1:16; preferably, the weight-to-volume ratio (g/mL) of the compound 1 in step (1-1) to the third low-polarity solvent in step (1-3) is 1:10-25; more preferably, the weight-to-volume ratio (a/mL) of the compound 1 in step (1-1) to the third low-polarity solvent in step (1-3) is 1:12-20; and more preferably, the weight-to-volume ratio (g/mL) of the compound 1 in step (1-1) to the third low-polarity solvent in step (1-3) is 1:15-17.

The invention also provides a preparation and purification method of a compound shown in the following formula:

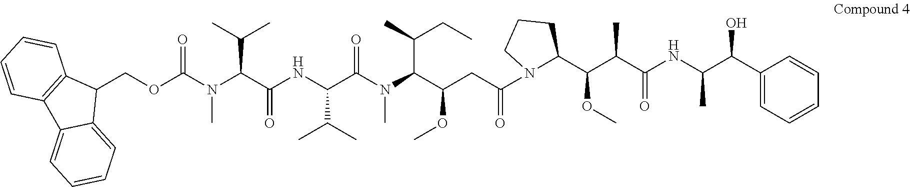 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCC1c2ccccc2-c2ccccc21)C(C)C]

the preparation route of the method is as follows:

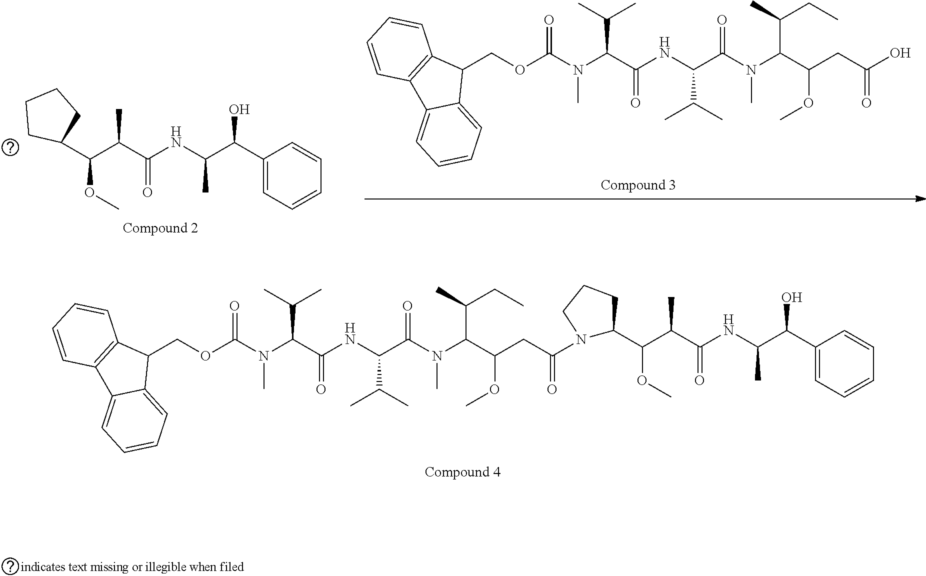

the method includes the following steps:
(2-1). dissolving a compound 2 and an appropriate amount of compound 3 in an eighth organic solvent to form a solution G;
(2-2). dissolving a second polypeptide condensing agent in an appropriate amount of ninth organic solvent to form a solution H, where the mole number of the second polypeptide condensing agent is larger than the mole number of the compound 3 in step (2-1);
(2-3). adding the solution H to the solution G to form a solution I;
(2-4). adding an appropriate amount of second organic base to the solution I for insulation reaction;
(2-5). after the reaction finishes, adding a sufficient amount of fourth low polarity solvent and purified water to the reaction system of step (2-4) for extraction, and collecting an organic phase; and
(2-6). washing the organic phase collected in step (2-5) with an appropriate amount of hydrochloric acid solution, purified water and sodium chloride solution successively, drying by anhydrous sodium sulfate, concentrating under reduced pressure, and drying to obtain a compound 4.

Further, the eighth organic solvent in step (2-1) is selected from DMF, DMA, DMSO, and DCM; and preferably, the eighth organic solvent is DMF.

Further, the weight-to-volume ratio (g/mL) of the compound 3 to the eighth organic solvent in step (2-1) is about 1:6; preferably, the weight-to-volume ratio (g/mL) of the compound 3 to the eighth organic solvent in step (2-1) is 1:5-10; more preferably, the weight-to-volume ratio (g/mL) of the compound 3 to the eighth organic solvent in step (2-1) is 1:5-8; and more preferably, the weight-to-volume ratio (g/mL) of the compound 3 to the eighth organic solvent in step (2-1) is 1:6-7.

Further, the second polypeptide condensing agent in step (2-2) is selected from HATU, DIC, DCC, EDC, HCTU, DEPBT, EEDQ and CDI; and preferably, the second polypeptide condensing agent in step (2-2) is HATU.

Further, the molar ratio of the compound 3 in step (2-1) to the second polypeptide condensing agent in step (2-2) is about 1:1.2; preferably, the molar ratio of the compound 3 in step (2-1) to the second polypeptide condensing agent in step (2-2) is 1:1.01-1.5; more preferably, the molar ratio of the compound 3 in step (2-1) to the second polypeptide condensing agent in step (2-2) is 1:1.1-1.4; and even more preferably, the molar ratio of the compound 3 in step (2-1) to the second polypeptide condensing agent in step (2-2) is 1:1.2-1.3.

Further, the ninth organic solvent in step (2-2) is selected from DMF, DMA, DMSO, and DCM; and preferably, the ninth organic solvent is DMF.

Further, the weight-to-volume ratio (g/mL) of the compound 3 in step (2-1) to the ninth organic solvent in step (2-2) is about 1:3; preferably, the weight-to-volume ratio (g/mL) of the compound 3 in step (2-1) to the ninth organic solvent in step (2-2) is 1:2-6; more preferably, the weight-to-volume ratio (g/mL) of the compound 3 in step (2-1) to the ninth organic solvent in step (2-2) is 1:2.5-4; and even more preferably, the weight-to-volume ratio (g/mL) of the compound 3 in step (2-1) to the ninth organic solvent in step (2-2) is 1:3-4.

Further, in step (2-3), the solution H is added dropwise to the solution G, and the internal temperature of the entire reaction system during the dropwise addition is 0-5° C.

Further, the second organic base in step (2-4) is one or more selected from N,N-diisopropylethylamine, triethylamine, and pyridine; and preferably, the second organic base in step (2-4) is N,N-diisopropylethyl amine.

Further, the molar ratio of the compound 3 in step (2-1) to the second organic base in step (2-4) is about 1:3; preferably, the molar ratio of the compound 3 in step (2-1) to the second organic base in step (2-4) is 1:2-5; more preferably, the molar ratio of the compound 3 in step (2-1) to the second organic base in step (2-4) is 1:2.5-4; and even more preferably, the molar ratio of the compound 3 in step (2-1) to the second organic base in step (2-4) is 1:3-4.

Further, in step (2-4), the second organic base is added dropwise to the solution I, and the temperature of the insulation reaction is 0-5° C.

Further, the fourth low-polarity solvent in step (2-5) is selected from methyl tert-butyl ether, ethyl acetate, dichloromethane, and tetrahydrofuran; preferably, the second low-polarity solvent in step (2-5) is methyl tert-butyl ether.

Further, the weight-to-volume ratio (g/mL) of the compound 3 in step (2-1) to the fourth low-polarity solvent and purified water in step (2-5) is about 1:20.2:20.2; preferably, the weight-to-volume ratio (g/mL) of the compound 3 in step (2-1) to the fourth low-polarity solvent and purified water in step (2-5) is 1:15-25:15-25; more preferably, the weight-to-volume ratio (g/mL) of the compound 3 in step (2-1) the fourth low-polarity solvent and purified water in step (2-5) is 1:20-24:20-24; and more preferably, the Height-to-volume ratio (giant) of the compound 3 in step (2-1) to the fourth low-polarity solvent and purified water in step (2-5) is 1:20-11:20-21.

Further, the volumes of the fourth low-polarity solvent and the purified water in step (2-5) are the same.

Further, the concentration of the hydrochloric acid solution in step (2-6) is about 0.05 mol/L; preferably, the concentration of the hydrochloric acid solution in step (2-6) is 0.02-0.08 mol/L; more preferably, the concentration of the hydrochloric acid solution in step (2-6) is 0.04-0.06 mol/L; and more preferably, the concentration of the hydrochloric acid solution in step (2-6) is 0.05 mol/L.

The invention also provides a preparation and purification method of a compound shown in formula (I):

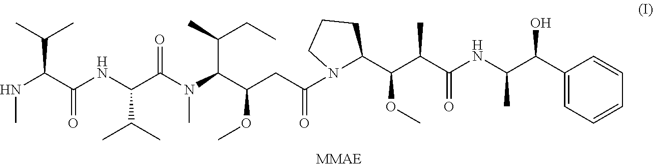 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

The preparation route of the method is as follows:

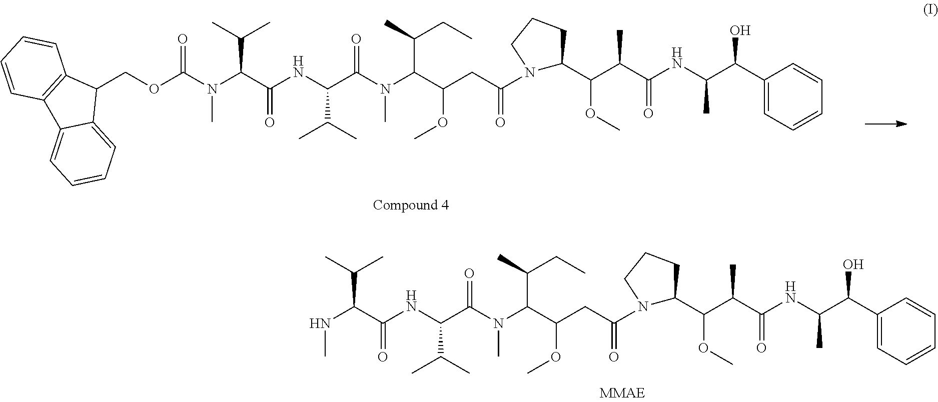 [SMILES: CC[C@H](C)C(C(CC(=O)N1CCC[C@H]1C(OC)[C@@H](C)C(=O)N[C@H](C)[C@@H](O)c1ccccc1)OC)N(C)C(=O)[C@@H](NC(=O)[C@H](C(C)C)N(C)C(=O)OCC1c2ccccc2-c2ccccc21)C(C)C ; CC[C@H](C)C(C(CC(=O)N1CCC[C@H]1C(OC)[C@@H](C)C(=O)N[C@H](C)[C@@H](O)c1ccccc1)OC)N(C)C(=O)[C@@H](NC(=O)[C@@H](NC)C(C)C)C(C)C]

The method includes the following steps:
(3-1). dissolving a compound 4 in a tenth organic solvent to form a solution J;
(3-2). adding a sufficient amount of diethylamine to the solution J for insulation reaction, removing a Fmoc protecting group;
(3-3). after the reaction finishes, adding an appropriate amount of eleventh organic solvent and purified water to the reaction system of step (3-2) for extraction, collecting an organic phase, drying by anhydrous sodium sulfate, and concentrating under reduced pressure;
(3-4). carrying out chromatographic purification on the concentrate under reduced pressure obtained in step (3-3) by an elution system of toluene:methanol, and concentrating the collected eluent under reduced pressure;
(3-5). after dissolving the concentrated product under reduced pressure obtained in step (3-4) with a twelfth organic solvent, filtering, and concentrating the filtrate under reduced pressure; and
(3-6). vacuum-drying the concentrated product under reduced pressure obtained in step (3-5) to obtain the MMAE.

Further, the tenth organic solvent in step (3-1) is selected from dichloromethane, acetonitrile, trichloromethane and carbon tetrachloride; and preferably, the tenth organic solvent in step (3-1) is dichloromethane.

Further, the weight-to-volume ratio (g/mL) of the compound 4 to the tenth organic solvent in step (3-1) is about 1:7; preferably, the weight-to-volume ratio (g/mL) of the compound 4 to the tenth organic solvent in step (3-1) is 1:4-10; and more preferably, the weight-to-volume ratio (g/mL) of the compound 4 to the tenth organic solvent in step (3-1) is 1:5-8.

Further, the weight-to-volume ratio (g/mL) of the compound 4 in step (3-1) to the diethylamine in step (3-2) is about 1:3.5; preferably, the weight-to-volume ratio (g/mL) of the compound 4 in step (3-1) to the diethylamine in step (3-2) is 1:3-5; and more preferably, the weight-to-volume ratio (g/mL) of the compound 4 in step (3-1) to the diethylamine in step (3-2) is 1:3-4.

Further, the diethylamine in step (3-2) is added dropwise, and in the dropwise addition process, the internal temperature is kept between 0° C. and 5° C.; and the temperature of the insulation reaction in step (3-2) is 20-30° C.

Further, the eleventh organic solvent in step (3-3) is selected from dichloromethane, trichloromethane, carbon tetrachloride and toluene; and preferably, the eleventh organic solvent in step (3-3) is dichloromethane.

Further, the weight-to-volume ratio (a/mL) of the compound 4 in step (3-1) to the eleventh organic solvent and purified water in step (3-3) is about 1:7:10; preferably, the weight-to-volume ratio (g/mL) of the compound 4 in step (3-1) to the eleventh organic solvent and purified water in step (3-3) is 1:5-10:5-15; and more preferably, the weight-to-volume ratio (g/mL) of the compound 4 in step (3-1) to the eleventh organic solvent and purified water in step (3-3) is 1:6-8:9-12.

Further, the silica gel used in the chromatographic purification in step (3-4) is 200-300 mesh silica gel; the elution system is toluene:methanol in a volume ratio (V/V) of 10-20:1; preferably, the elution system is firstly toluene:methanol in a volume ratio (V/V) of about 20:1; and when TLC detects that only the product is visible, the elution system is changed to toluene:methanol in a volume ratio (V/V) of about 10:1.

Further, the developing agent of the TLC detection is toluene:methanol in a volume ratio (V/V) of about 5:1.

Further, the twelfth organic solvent in step (3-5) is selected from methanol, toluene and acetonitrile; and preferably, the twelfth organic solvent in step (3-5) is methanol.

Further, the weight-to-volume ratio (g/mL) of the compound 4 in step (3-1) to the twelfth organic solvent in step (3-5) is about 1:3-10.

Further, the process in step (3-5) may be repeated 1-5 times.

The preparation and purification process of MMAE provided by the present invention has mild synthesis and purification conditions, can effectively prevent the change of product chirality caused by excessively high temperature, greatly reduces the generation of degradation impurities, improves the purity of the product, and increases the yield of the product. In addition, the preparation and purification process provided by the present invention has good stability and is more suitable for scale-up production. The MMAE prepared by the preparation and purification process provided by the present invention has purity of higher than 99%, and can perfectly meet clinical drug requirements.

### BRIEF DESCRIPTION OF FIGURES

FIG. 1 is the chromatogram of the compound 2.

FIG. 2 is the chromatogram of the compound 4.

FIG. 3 is the chromatogram of the compound MMAE.

### DETAILED DESCRIPTION

The technical solutions of the present invention are further described in non-limiting detail below in conjunction with specific embodiments. It should be pointed out that the following embodiments are only to illustrate the technical concept and characteristics of the present invention, and its purpose is to enable those skilled in the art to understand the content of the present invention and implement it accordingly, and cannot limit the protection scope of the present invention. All equivalent changes or modifications made according to the spirit of the present invention should be included within the protection scope of the present invention.

### Example 1 Preparation and Purification of Compound 2

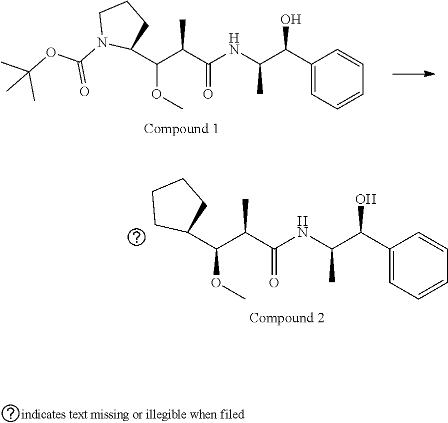

115.05 g of compound 1 (273.58 mmol) and 230 mL of dichloromethane (V_{dichloromethane}/W_{compound 1}=2.0) were added to the reaction flask. The internal temperature was controlled to be 0-5° C., 690 mL of 4 mol/L HCl-1,4-dioxane solution (V_{4 mol/L HCL-1,4-dioxane solution}/W_{compound 1}=6.0) was added dropwise. After the dropwise addition, the temperature was raised to 10° C., and the temperature was kept at 10-15° C. for reaction for 1 h. After sampling of ultra-high performance liquid chromatography to monitor the reaction, UPLC was sampled to monitor the reaction every 0.5 h. and samples were taken to detect the remaining amount of the compound 1. When the remaining amount of the compound 1 was less than 1.0%, the reaction was considered complete.

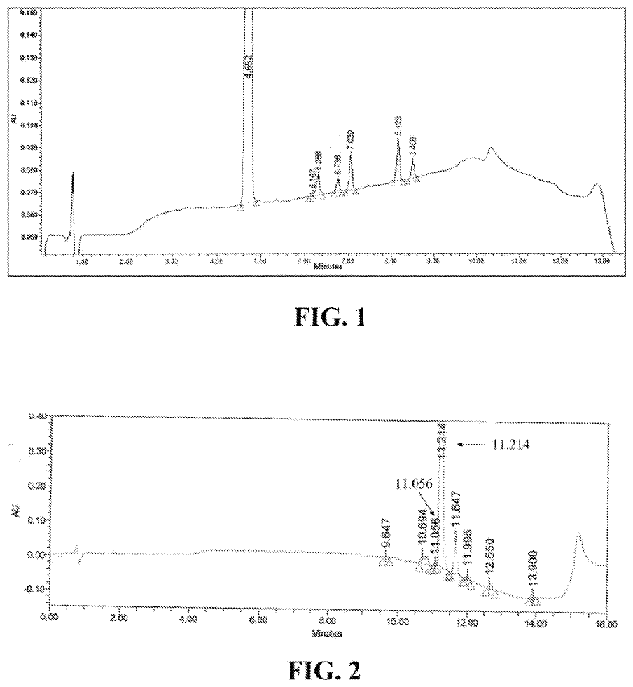

After the completion of the reaction, the above reaction solution was slowly poured into a reaction flask containing 1840 mL, of n-hexane (Vₙ₋ₕₑₓₐₙₑ/W_{compound 1}=16:0) under stirring (n-hexane can be cooled to 0-5° C. in advance). Stirring was continued for 30 min and the supernatant was decanted. The solid in the reaction flask was vacuum-dried with a diaphragm vacuum pump at 30-35° C. for 1±0.5 h. An oil pump was used continuously for drying under vacuum at room temperature (18-26° C.) for not less than 12 hours, and the compound 2 (with yield of 119%, purity of 95.4%, and maximum single impurity of 1.4%) was obtained when the weight did not change. Its chromatogram was shown in FIG. 1.

### Example 2 Preparation and Purification of Compound 4

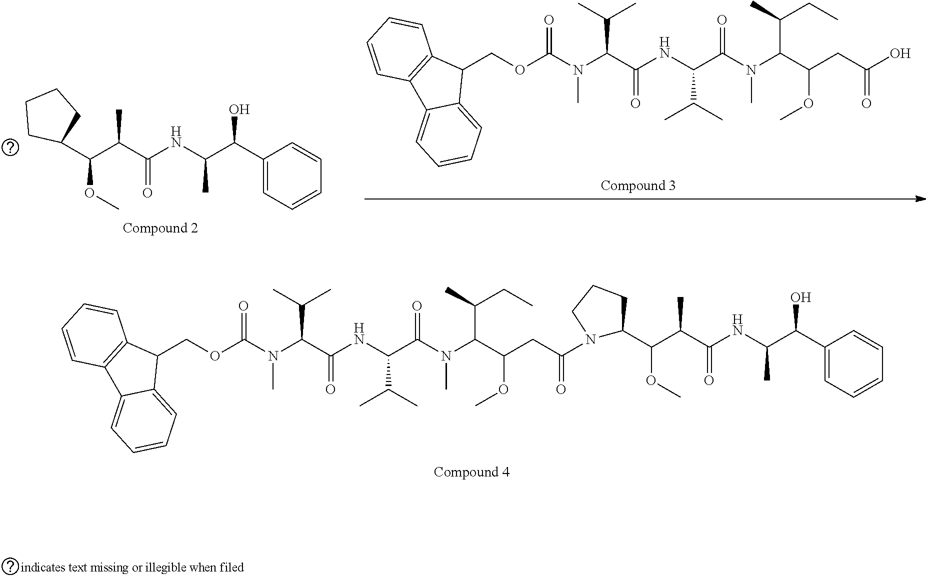

114.17 g of compound 2 (301.52 mmol), 192.31 g of compound 3 (301.52 mmol) and 1160 mL of DMF (V_{DMF}/W of_{compound 3}=6.0) were sequentially added to the reaction flask, another 139.04 g of HAM (365.67 mmol) was dissolved in 580 mL of DMF (W_{DMF}/W_{compound 3}=3.0), and a DMF solution of HAW was formed. The internal temperature was controlled at 0-5° C., and the DMF solution of HATU was added dropwise to the DMF solution of the compound 2 and compound 3. After the dropwise addition was completed, the internal temperature was controlled at 0-5° C. for 20±2 min. Then, 118.07 g of N,N-diisopropylethylamine (913.50 mmol) was added dropwise under the control of the internal temperature at 0-5° C.; after the dropwise addition, the temperature was kept at 0-5° C. for reaction for 1 h. Sampling was carried out for UPLC to monitor the reaction, and then sampling was carried out every 0.5 h to detect the remaining amount of the compound 3. When the remaining amount of the compound 3 was less than 6.0%, the reaction was considered complete.

After the completion of the reaction, the reaction solution was transferred to a 30 L glass reactor, and 3900 mL of methyl tert-butyl ether and 3900 mL of purified water (cooled to in advance) were successively added for extraction, and organic phases were separated. The aqueous phase was extracted twice more with 3900 mL of methyl tert-butyl ether. The organic phases were combined.

The above organic phase was washed with 3900 mL of 0.05 mold, hydrochloric acid solution (cooled to 0° C. in advance), and the organic phase was collected. The organic phase was washed with 3900 mL of purified water (V_{purified water}/W_{compound 3}=20.2) (cooled to 0° C. in advance), and the organic phase was collected. Then the organic phase was washed with 3900 mL of 30% aqueous sodium chloride solution, and the organic phase was collected. Then, the organic phase was stirred and dried with 388.48 g of anhydrous sodium sulfate for 0.5 h. The desiccant was filtered off, the filter cake was washed with 1950 mL of methyl tert-butyl ether, and the filtrates were combined, and then concentrated under reduced pressure at 30-35° C. to foam. The oil pump was vacuum-dried for at least 1 h, and the compound 4 (with yield of 112%, purity of 88.6%, and maximum single impurity of 5.6%) was obtained when the weight did not change. Its chromatogram is shown in FIG. 2.

### Example 3 Preparation and Purification of MMAE

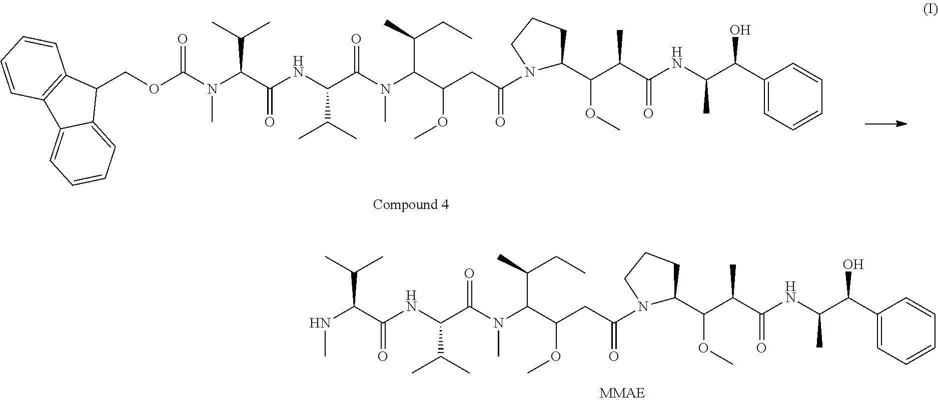 [SMILES: CC[C@H](C)C(C(CC(=O)N1CCC[C@H]1C(OC)[C@@H](C)C(=O)N[C@H](C)[C@@H](O)c1ccccc1)OC)N(C)C(=O)[C@@H](NC(=O)[C@H](C(C)C)N(C)C(=O)OCC1c2ccccc2-c2ccccc21)C(C)C ; CC[C@H](C)C(C(CC(=O)N1CCC[C@H]1C(OC)[C@@H](C)C(=O)N[C@H](C)[C@@H](O)c1ccccc1)OC)N(C)C(=O)[C@@H](NC(=O)[C@@H](NC)C(C)C)C(C)C]

307.22 g of compound 4 (326.75 mmol) and 1900 mL of dichloromethane were added to the reaction flask. The internal temperature was controlled at 0-5° C., and 950 mL of diethylamine was added dropwise. After the dropwise addition, the temperature was raised to 20° C. to start the timing reaction, and the temperature was kept at 20-30° C. for reaction for 10 h. Sampling was carried out for UPLC to monitor the reaction, and then sampling was carried out every 1 h for UPLC to monitor the reaction, sampling was carried out to detect the remaining amount of the compound 4. When the remaining amount of the compound 4 was less than 1.0%, the reaction was considered complete.

After the reaction was completed, the reaction solution was transferred to a 30 L glass reactor, 1900 mL of dichloromethane was added, washed twice with 2700 mL of purified water (cooled to 0° C. in advance), and the organic phase was separated. The organic phase was stirred and dried with 542.04 g of anhydrous sodium sulfate for 0.5 h, the desiccant was filtered off, the filter cake was washed with 810 mL of dichloromethane, and the filtrates were combined. The filtrate was concentrated under reduced pressure at 30-35° C. to foam. An oil pump was used for vacuum-drying at room temperature (18-26° C.) for at least 1 h, and crude MMAE was obtained when the weight did not change.

After the chromatography column was cleaned, the column was packed: 13986.14 g of silica gel (200-300 meshes) and 40 L of toluene were stirred to a uniform fluid state, and then transferred to the chromatography column in batches (standing for 1 h), and 5-8 cm of the toluene was kept at the top of the silica gel, and the toluene on the silica surface was drained.

262.17 g of crude MMAE was dissolved in 500 mL of dichloromethane; the dichloromethane solution of crude MMAE was slowly poured into a sieve, and after adding, the liquid on the sample surface was drained to ensure that the upper surface of the sample is flat, and 2896.58 g of anhydrous sodium sulfate was added to the top end of the silica column. A 30 L double-layer glass reactor was used to prepare an eluent, the eluent was cooled to 2-8° C., and eluted with a 235.2 L of toluene:methanol=20:1 (V/V) system firstly, and detection was not carried out until the color band completely flew out. After the color band flew out, thin layer chromatography (TLC) detection started (developing agent was Vₜₒₗᵤₑₙₑ:Vₘₑₜₕₐₙₒₗ=5:1, iodine was used for color development), when only the product was visible, 140.8 L of toluene:methanol=10:1 (VAT) was used instead for continuous elution until the product completely flew out (no product was detected by TLC).

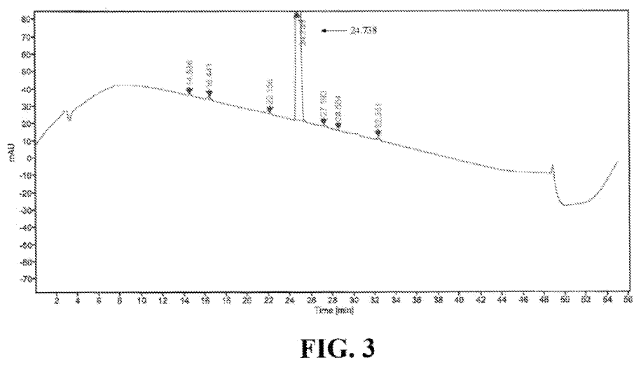

The pure fractions of MMAE were combined and concentrated under reduced pressure at 35-40° C. to foam. After dissolving with 1200 mL of methanol, the product was filtered and concentrated under reduced pressure at 35-40° C.; the operation was repeated twice. After drying at 40-45° C. for 10-18 h under oil pump vacuum, grinding was carried out several times until a uniform powder is obtained. After continuous drying for a total of 36 hours, sampling started every 6-12 h to detect methanol and toluene solvent residues. When methanol residues were less than or equal to 0,200% and toluene residues were less than or equal to 0.089%, drying was stopped to obtain purified MMAE with yield of 71.79%, purity of 99.8% and single impurity of 0.2%. Its chromatogram is shown in FIG. 3.

The present invention has been exemplified by various specific embodiments. However, those of ordinary skill in the art can understand that the present invention is not limited to each specific embodiment, and those of ordinary skill can make various changes or modifications within the scope of the present invention, and various technical features mentioned in various places in this specification can be combined with each other without departing from the spirit and scope of the present invention. Such modifications and variations are within the scope of the present invention.

1. A preparation and purification method of a compound shown in formula (I): 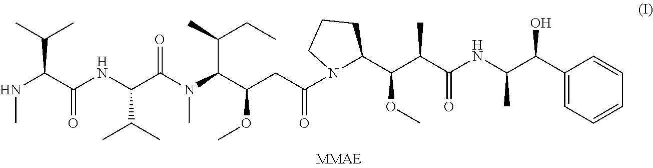 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]
the preparation route of the method being as follows: 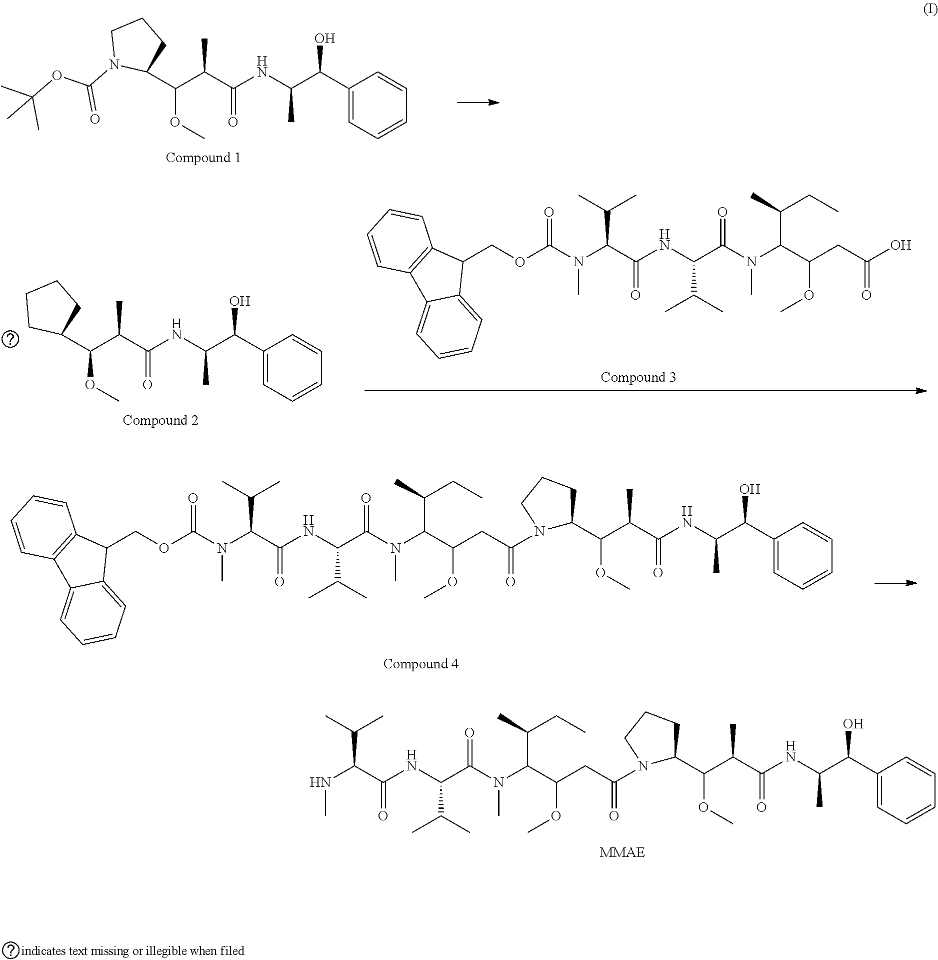
Wherein the method comprises the following steps:
(1) dissolving a compound 1 in an appropriate amount of a first organic solvent to form a solution A;
(2) adding a sufficient amount of HCl-1,4-dioxane solution to the solution A for insulation reaction, removing a Boc protecting group;
(3) after the reaction finishes, pouring the reaction solution of step (2) into a sufficient amount of first low-polarity solvent, discarding the filtrate after stirring, and solid residues being compound 2 after drying;
(4) dissolving the obtained compound 2 and an appropriate amount of compound 3 in a second organic solvent to form a solution B;
(5) dissolving a first polypeptide condensing agent in an appropriate amount of third organic solvent to form a solution C, wherein the mole number of the first polypeptide condensing agent is larger than the mole number of the compound 3 of step (4);
(6) adding the solution C to the solution B to form a solution D;
(7) adding an appropriate amount of first organic base to the solution D for insulation reaction;
(8) after the reaction of step (7) finishes, adding a sufficient amount of second low polarity solvent and purified water to the reaction system of step (7) for extraction, and collecting an organic phase;
(9) washing the organic phase collected in step (8) with an appropriate amount of hydrochloric acid solution, purified water and sodium chloride solution successively, drying by anhydrous sodium sulfate, concentrating under reduced pressure, and drying to obtain a compound 4;
(10) dissolving the compound 4 in a fourth organic solvent to form a solution E;
(11) adding a sufficient amount of diethylamine to the solution E for insulation reaction, removing a Fmoc protecting group;
(12) after the reaction finishes, adding an appropriate amount of fifth organic solvent and purified water to the reaction system of step (11) for extraction, collecting an organic phase, drying by anhydrous sodium sulfate, and concentrating under reduced pressure;
(13) carrying out chromatographic purification on the concentrate under reduced pressure obtained in step (12) by an elution system of toluene:methanol, and concentrating the collected eluent under reduced pressure;
(14) after dissolving the concentrated product under reduced pressure obtained in step (13) with a sixth organic solvent, filtering, and concentrating the filtrate under reduced pressure; and
(15) vacuum-drying the concentrated product under reduced pressure obtained in step (14) to obtain the MMAE.

2. The method according to claim 1, wherein the first organic solvent in step 1 is selected from dichloromethane, trichloromethane and carbon tetrachloride; and preferably, the first organic solvent in step 1 is dichloromethane.

3. The method according to claim 1, wherein the weight-to-volume ratio (g/mL) of the compound 1 to the first organic solvent in step 1 is about 1:2; preferably, the weight-to-volume ratio (g/mL) of the compound 1 to the first organic solvent in step 1 is 1:1-3; and more preferably, the weight-to-volume ratio (g/mL) of the compound 1 to the first organic solvent in step 1 is 1:1.5-2.5.

4. The method according to claim 1, wherein the concentration of the HCl-1,4-dioxane solution in step (2) is about 4 mol/L; preferably, the concentration of the HCl-1,4-dioxane solution in step (2) is about 3-7 mol/L; and more preferably, the concentration of the HCl-1,4-dioxane solution in step (2) is 3.5-4.5 mol/L.

5. The method according to claim 1, wherein the weight-to-volume ratio (g/mL) of the compound 1 in step 1 to the HCl-1,4-dioxane solution in step (2) is about 1:6; preferably, the weight-to-volume ratio (g/mL) of the compound 1 in step 1 to the HCl-1,4-dioxane solution in step (2) is 1:4-8; more preferably, the weight-to-volume ratio (g/mL) of the compound 1 in step 1 to the HCl-1,4-dioxane solution in step (2) is 1:5-7; and inure preferably, the weight-to-volume ratio (g/mL) of the compound 1 in step 1 to the HCl-1,4-dioxane solution in step (2) is 1:6.

6. The method according to claim 1, wherein the HCl-1,4-dioxane solution in step (2) is added dropwise, and the internal temperature of the reaction system is maintained between −5° C.-5° C. during the dropwise addition.

7. The method according to claim 1, wherein the temperature of the insulation reaction in step (2) is 10-15° C.

8. The method according to claim 1, wherein the first low-polarity solvent in step (3) is selected from n-hexane, petroleum ether, and n-heptane; and preferably, the first low-polarity solvent is selected from n-hexane.

9. The method according to claim 1, wherein the weight-to-volume ratio (g/mL) of the compound 1 in step 1 to the first low-polarity solvent in step (3) is about 1:16; preferably, the weight-to-volume ratio (g/mL) of the compound 1 in step 1 to the first low-polarity solvent in step (3) is 1:10-25; more preferably, the weight-to-volume ratio (g/mL) of the compound 1 in step 1 to the first low-polarity solvent in step (3) is 1:12-20; and more preferably, the weight-to-volume ratio (g/mL) of the compound 1 in step 1 to the first low-polarity solvent in step (3) is 1:15-17.

10. The method according to claim 1, wherein the second organic solvent in step (4) is selected from DMF, DMA, DMSO and DCM; preferably, the second organic solvent is DMF; and further preferably, in step (4), the molar amounts of the compound 2 and the compound 3 are the same.

11. The method according to claim 1, wherein the weight-to-volume ratio (g/mL) of the compound 3 to the second organic solvent in step (4) is about 1:6; preferably, the weight-to-volume ratio (g/mL) of the compound 3 to the second organic solvent in step (4) is 1:5-10; more preferably, the weight-to-volume ratio (g/mp) of the compound 3 to the second organic solvent in step (4) is 1:5-8; and even more preferably, the weight-to-volume ratio (g/mL) of the compound 3 to the second organic solvent in step (4) is 1:6-7.

12. The method according to claim 1, wherein the first polypeptide condensing agent in step (5) is selected from HATU, DIC, DCC, FDC, HCTU, DEPBT, EEDQ and CDI, and preferably, the first polypeptide condensing agent in step (5) is HATU.

13. The method according to claim 1, wherein the molar ratio of the compound 3 in step (4) to the first polypeptide condensing agent in step (5) is about 1.1.2; preferably, the molar ratio of the compound 3 in step (4) to the first polypeptide condensing agent in step (5) is 1:1.01-1.5; more preferably, the molar ratio of the compound 3 in step (4) to the first polypeptide condensing agent in step (5) is 1:1.1-1.4; and even more preferably, the molar ratio of the compound 3 in step (4) to the first polypeptide condensing agent in step (5) is 1:1.2-1.3.

14. The method according to claim 1, wherein the third organic solvent in step (5) is selected from DMF, DMA, DMSO, and DCM; and preferably, the third organic solvent is DMF.

15. The method according to claim 1, wherein the weight-to-volume ratio (g/mL) of the compound 3 in step (4) to the third organic solvent in step (5) is about 1:3; preferably, the weight-to-volume ratio (g/mL) of the compound 3 in step (4) to the third organic solvent in step (5) is 1:2-6; more preferably, the weight-to-volume ratio (g/mL) of the compound 3 in step (4) to the third organic solvent in step (5) is 1:2.5-4; and more preferably, the weight-to-volume ratio (g/mL) of the compound 3 in step (4) to the third organic solvent in step (5) is 1:3-4.

16. The method according to claim 1, wherein in step (6) the solution C is added dropwise to the solution B, and the internal temperature of the entire reaction system during the dropwise addition is 0-5° C.

17. The method according to claim 1, wherein the first organic base in step (7) is one or more selected from N,N-diisopropylethylamine, triethylamine; and pyridine; and preferably, the first organic base in step (7) is N,N-diisopropylethylamine.

18. The method according to claim 1, wherein the molar ratio of the compound 3 in step (4) to the first organic base in step (7) is about 1:3; preferably, the molar ratio of the compound 3 in step (4) to the first organic base in step (7) is 1:2-5; more preferably, the molar ratio of the compound 3 in step (4) to the first organic base in step (7) is 1:2.5-4; and even more preferably, the molar ratio of the compound 3 in step (4) to the first organic base in step (7) is 1:3-4.

19. The method according to claim 1, wherein the first organic base in step (7) is added dropwise to the solution D, and the temperature of the insulation reaction is 0-5° C.

20. The method according to claim 1, wherein the second low-polarity solvent in step (8) is selected from methyl tert-butyl ether, ethyl acetate, dichloromethane, and tetrahydrofuran, and preferably, the second low-polarity solvent in step (8) is methyl tert-butyl ether.

21. The method according to claim 1, wherein the weight-to-volume ratio (g/mL) of the compound 3 in step (4) to the second low-polarity solvent and purified water in step (8) about 1:20.2:20.2; preferably, the weight-to-volume ratio (g/mL) of the compound 3 in step (4) to the second low-polarity solvent and purified water in step (8) is 1:15-25:15-25; more preferably, the weight-to-volume ratio (g/mL) of the compound 3 in step (4) to the second low-polarity solvent and purified water in step (8) is 1:20-24:20-24; and more preferably, the weight-to-volume ratio (g/mL) of the compound 3 in step (4) to the second low-polarity solvent and purified water in step (8) is 1:20-21:20-21.

22. The method according to claim 1, wherein the volumes of the second low-polarity solvent and the purified water in step (8) are the same.

23. The method according to claim 1, wherein the concentration of the hydrochloric acid solution in step (9) is about 0.05 mol/L; preferably, the concentration of the hydrochloric acid solution in step (9) is 0.02-0.08 mol/L; more preferably, the concentration of the hydrochloric acid solution in step (9) is 0.04-0.06 mol/L; and more preferably, the concentration of the hydrochloric acid solution in step (9) is 0.05 mol/L.

24. The method according to claim 1, wherein the concentration of the sodium chloride solution in step (9) is about 30%; and preferably, the concentration of the sodium chloride solution in step (9) is 20%-40%.

25. The method according to claim 1, wherein the volume of the hydrochloric acid solution, the purified water and the sodium chloride solution in step (9) is equal to the volume of the second low-polarity solvent in step (8).

26. The method according to claim 1, wherein the fourth organic solvent in step (10) is selected from dichloromethane, acetonitrile, trichloromethane and carbon tetrachloride; and preferably, the fourth organic solvent in step (10) is dichloromethane.

27. The method according to claim 1, wherein the weight-to-volume ratio (g/mL) of the compound 4 to the fourth organic solvent in step (10) is about 1:7; preferably, the weight-to-volume ratio (g/mL) of the compound 4 to the fourth organic solvent in step (10) is 1:4-10; and more preferably, the weight-to-volume ratio (g/mL) of the compound 4 to the fourth organic solvent in step (10) is 1:5-8.

28. The method according to claim 1, wherein the weight-to-volume ratio (g/mL) of the compound 4 in step (10) to the diethylamine in step (11) is about 1:3.5; preferably, the weight-to-volume ratio (g/mL) of the compound 4 in step (10) to the diethylamine in step (11) is 1:3-5; and more preferably, the weight-to-volume ratio (g/mL) of the compound 4 in step (10) to the diethylamine in step (11) is 1:3-4.

29. The method according to claim 1, wherein the diethylamine in step (11) is added dropwise to the solution E, and in the dropwise addition process, the internal temperature of the solution is kept between 0-5° C.; and the temperature of the insulation reaction in step (11) is 20-30° C.

30. The method according to claim 1, wherein the fifth organic solvent in step (12) is selected from dichloromethane, trichloromethane, carbon tetrachloride and toluene; and preferably, the fifth organic solvent in step (12) is dichloromethane.

31. The method according to claim 1, wherein the weight-to-volume ratio (g/mL) of the compound 4 in step (10) to the fifth organic solvent and purified water in step (12) is about 1.7:10; preferably, the weight-to-volume ratio (g/mL) of the compound 4 in step (10) to the fifth organic solvent and purified water in step (12) is 1:5-10:5-15; and more preferably, the weight-to-volume ratio (g/mL) of the compound 4 in step (10) to the fifth organic solvent and purified water in step (12) is 1:6-8:9-12.

32. The method according to claim 1, wherein the silica gel used in the chromatographic purification in step (13) is 200-300 mesh silica gel; the elution system is toluene:methanol in a volume ratio (V/V) of 10-20:1; preferably, the elution system is firstly toluene:methanol in volume ratio (V/V) of about 20:1; and when TLC detects that only the product is visible, the elution system is changed to toluene:methanol in a volume ratio (V/V) of about 10:1.

33. The method according to claim 32, wherein the developing agent of the TLC detection is toluene:methanol in a volume ratio (V/V) of about 5:1.

34. The method according to claim 1, wherein the sixth organic solvent in step (14) is selected from methanol, toluene and acetonitrile; and preferably, the sixth organic solvent in step (14) is methanol.

35. The method according to claim 1, wherein the weight-to-volume ratio (g/mL) of the compound 4 in step (10) to the sixth organic solvent in step (14) is about 1:3-10.

36. The method according to claim 1, wherein the process of step (14) can be repeated 1-5 times.

37. A preparation and purification method of a compound shown in the following formula: 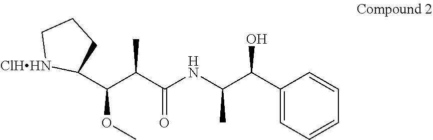
the preparation route of the method being as follows: 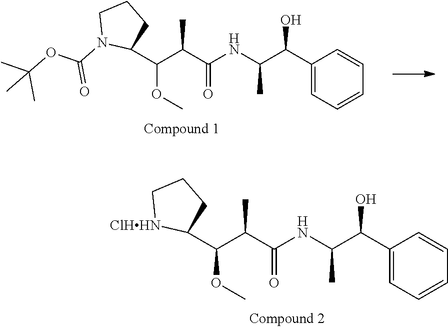
wherein the method comprises the following steps:
(1-1). dissolving a compound 1 in an appropriate amount of seventh organic solvent to form a solution F;
(1-2). adding a sufficient amount of HCl-1,4-dioxane solution to the solution F for insulation reaction, removing a Boc protecting group; and
(1-3). after the reaction finishes, pouring the reaction solution of step (1-2) into a sufficient amount of third low-polarity solvent, discarding a filtrate after stirring, and solid residues being a compound 2 after drying.

38. The method according to claim 37, wherein the seventh organic solvent in step (1-1) is selected from dichloromethane, trichloromethane and carbon tetrachloride; and preferably, the seventh organic solvent in step (1-1) is dichloromethane.

39. The method according to claim 37, wherein the weight-to-volume ratio (g/mL) of the compound 1 to the seventh organic solvent in step (1-1) is about 1:2; preferably, the weight-to-volume ratio (g/mL) of the compound 1 to the seventh organic solvent in step (1-1) is 1:1-3; and more preferably, the weight-to-volume ratio (g/mL) of the compound 1 to the seventh organic solvent in step (1-1) is 1:1.5-2.5.

40. The method according to claim 37, wherein the concentration of the HCl-1,4-dioxane solution in step (1-2) is about 4 mol/L; preferably, the concentration of the HCl-1,4-dioxane solution in step (1-2) is about 3-7 mol/L; and more preferably, the concentration of the HCl-1,4-dioxane solution in step (1-2) is 3.5-4.5 mol/L.

41. The method according to claim 37, wherein the weight-to-volume ratio (g/mL) of the compound 1 in step (1-1) to the HCl-1,4-dioxane solution in step (1-2) is about 1:6; preferably, the weight-to-volume ratio (g/mL) of the compound 1 in step (1-1) to the HCl-1,4-dioxane solution in step (1-2) is 1:4-8; more preferably, the weight-to-volume ratio (g/mL) of the compound 1 in step (1-1) to the HCl-1,4-dioxane solution in step (1-2) is 1:5-7; and even more preferably, the weight-to-volume ratio (g/mL) of the compound 1 in step (1-1) to the HCl-1,4-dioxane solution in step (1-2) is 1:6.

42. The method according to claim 37, wherein the HCl-1,4-dioxane solution in step (1-2) is added dropwise, and the internal temperature of the reaction system is maintained between −5-5° C. during the dropwise addition.

43. The method according to claim 37, wherein the temperature of the insulation reaction in step (1-2) is 10-15° C.

44. The method according to claim 37, wherein the third low-polarity solvent in step (1-3) is selected from n-hexane, petroleum ether, and n-heptane; and preferably, the third low-polarity solvent in step (1-3) is selected from n-hexane.

45. The method according to claim 37, wherein the weight-to-volume ratio (g/mL) of the compound 1 in step (1-1) to the third low-polarity solvent in step (1-3) is about 1:16; preferably, the weight-to-volume ratio (g/mL) of the compound 1 in step (1-1) to the third low-polarity solvent in step (1-3) is 1:10-25; more preferably, the weight-to-volume ratio (g/mL) of the compound 1 in step (1-1) to the third low-polarity solvent in step (1-3) is 1:12-20; and more preferably, the weight-to-volume ratio (g/mL) of the compound 1 in step (1-1) to the third low-polarity solvent in step (1-3) is 1:15-17.

46. A preparation and purification method of a compound shown in the following formula: 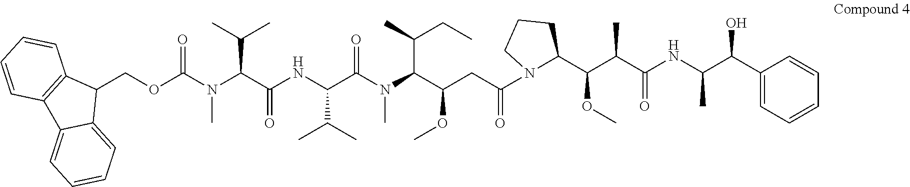 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCC1c2ccccc2-c2ccccc21)C(C)C]
the preparation route of the method being as follows: 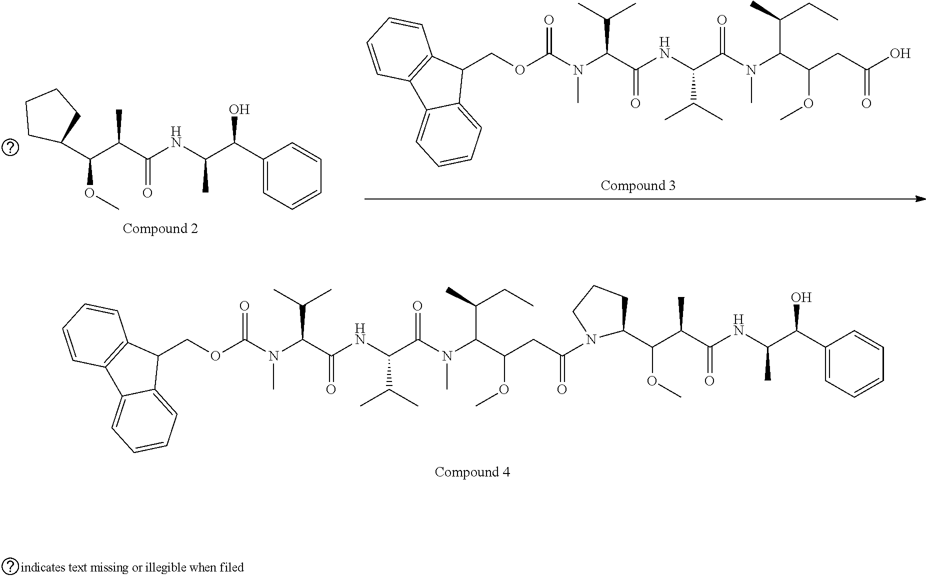
wherein the method comprises the following steps:
(2-1). dissolving a compound 2 and an appropriate amount of compound 3 in an eighth organic solvent to form a solution G;
(2-2). dissolving a second polypeptide condensing agent in an appropriate amount of ninth organic solvent to form a solution H, wherein the mole number of the second polypeptide condensing agent is larger than the mole number of the compound 3 in step (2-1);
(2-3). adding the solution H to the solution G to form a solution 1;
(2-4). adding an appropriate amount of second organic base to the solution 1 for insulation reaction;
(2-5). after the reaction finishes, adding a sufficient amount of fourth low polarity solvent and purified water to the reaction system of step (2-4) for extraction, and collecting an organic phase; and
(2-6). washing the organic phase collected in step (2-5) with an appropriate amount of hydrochloric acid solution, purified water and sodium chloride solution successively, drying by anhydrous sodium sulfate, concentrating under reduced pressure, and drying to obtain a compound 4.

47. The method according to claim 46, wherein the eighth organic solvent in step (2-1) is selected from DMF, DMA, DMSO, and DCM; and preferably, the eighth organic solvent is DMF.

48. The method according to claim 46, wherein the weight-to-volume ratio (g/mL) of the compound 3 to the eighth organic solvent in step (2-1) is about 1:6; preferably, the weight-to-volume ratio (g/mL) of the compound 3 to the eighth organic solvent in step (2-1) is 1:5-10; more preferably, the weight-to-volume ratio (g/mp) of the compound 3 to the eighth organic solvent in step (2-1) is 1:5-8; and more preferably, the weight-to-volume ratio (g/mL) of the compound 3 to the eighth organic solvent in step (2-1) is 1:6-7.

49. The method according to claim 46, wherein the second polypeptide condensing agent in step (2-2) is selected from HAM, DIC, DCC, EDC, HCTU, DEPBT, EEDQ and CDI; and preferably, the second polypeptide condensing agent in step (2-2) is HATU.

50. The method according to claim 46, wherein the molar ratio of the compound 3 in step (2-1) to the second polypeptide condensing agent in step (2-2) is about 1:1.2; preferably, the molar ratio of the compound 3 in step (2-1) to the second polypeptide condensing agent in step (2-2) is 1:1.01-1.5; more preferably, the molar ratio of the compound 3 in step (2-1) to the second polypeptide condensing agent in step (2-2) is 1:1.1-1.4; and even more preferably, the molar ratio of the compound 3 in step (2-1) to the second polypeptide condensing agent in step (2-2) is 1:1.2-1.3.

51. The method according to claim 46, wherein the ninth organic solvent in step (2-2) is selected from DMF, DMA, DMSO, and DCM; and preferably, the ninth organic solvent is DMF.

52. The method according to claim 46, wherein the weight-to-volume ratio (g/mL) of the compound 3 in step (2-1) to the ninth organic solvent in step (2-2) is about 1:3; preferably, the weight-to-volume ratio (g/mL) of the compound 3 in step (2-1) to the ninth organic solvent in step (2-2) is 1:2-6; more preferably, the weight-to-volume ratio (g/mL) of the compound 3 in step (2-1) to the ninth organic solvent in step (2-2) is 1:2.5-4; and even more preferably, the weight-to-volume ratio (g/mL) of the compound 3 in step (2-1) to the ninth organic solvent in step (2-2) is 1:3-4.

53. The method according to claim 46, wherein in step (2-3), the solution H is added dropwise to the solution (1, and the internal temperature of the entire reaction system during the dropwise addition is 0-5° C.

54. The method according to claim 46, wherein the second organic base in step (2-4) is one or more selected from N,N-diisopropylethylamine, triethylamine, and pyridine; and preferably, the second organic base in step (2-4) is N,N-diisopropylethylamine.

55. The method according to claim 46, wherein the molar ratio of the compound 3 in step (2-1) to the second organic base in step (2-4) is about 1:3; preferably, the molar ratio of the compound 3 in step (2-1) to the second organic base in step (2-4) is 1:2-5; more preferably, the molar ratio of the compound 3 in step (2-1) to the second organic base in step (2-4) is 1:2.5-4; and even more preferably, the molar ratio of the compound 3 in step (2-1) to the second organic base in step (2-4) is 1:3-4.

56. The method according to claim 46, wherein in step (2-4), the second organic base is added dropwise to the solution 1, and the temperature of the insulation reaction is 0-5° C.

57. The method according to claim 46, wherein the fourth low-polarity solvent in step (2-5) is selected from methyl tert-butyl ether, ethyl acetate, dichloromethane, and tetrahydrofuran; and preferably, the fourth low-polarity solvent in step (2-5) is methyl tert-butyl ether.

58. The method according to claim 46, wherein the weight-to-volume ratio (g/mL) of the compound 3 in step (2-1) to the fourth low-polarity solvent and purified water in step (2-5) is about 1:20.2:20.2; preferably, the weight-to-volume ratio (g/mL) of the compound 3 in step (2-1) to the fourth low-polarity solvent and purified water in step (2-5) is 1:15-25:15-25; more preferably, the weight-to-volume ratio (g/mL) of the compound 3 in step (2-1) to the fourth low-polarity solvent and purified water in step (2-5) is 1:20-24:20-24, and more preferably, the weight-to-volume ratio (g/ml) of the compound 3 in step (2-1) to the fourth low-polarity solvent and purified water in step (2-5) is 1:20-21:20-21.

59. The method according to claim 46, wherein the volume of the fourth low-polarity solvent and the volume of the purified water in step (2-5) are the same.

60. The method according to claim 46, wherein the concentration of the hydrochloric acid solution in step (2-6) is about 0.05 mol/L; preferably, the concentration of the hydrochloric acid solution in step (2-6) is 0.02-0.08 mol/L; more preferably, the concentration of the hydrochloric acid solution in step (2-6) is 0.04-0.06 mol/L; and more preferably, the concentration of the hydrochloric acid solution in step (2-6) is 0.05 mol/L.

61. A preparation and purification method of a compound shown in formula (I): 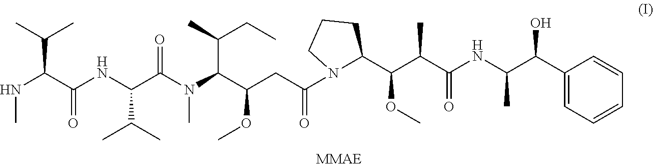 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]
the preparation route of the method being as follows: 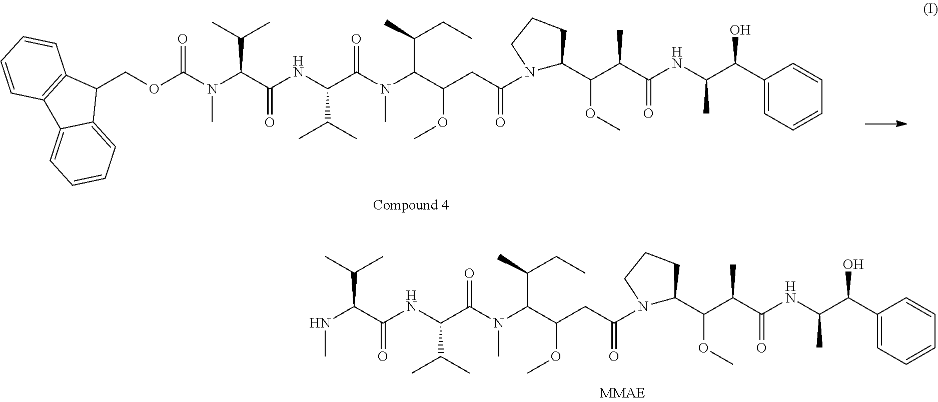 [SMILES: CC[C@H](C)C(C(CC(=O)N1CCC[C@H]1C(OC)[C@@H](C)C(=O)N[C@H](C)[C@@H](O)c1ccccc1)OC)N(C)C(=O)[C@@H](NC(=O)[C@H](C(C)C)N(C)C(=O)OCC1c2ccccc2-c2ccccc21)C(C)C ; CC[C@H](C)C(C(CC(=O)N1CCC[C@H]1C(OC)[C@@H](C)C(=O)N[C@H](C)[C@@H](O)c1ccccc1)OC)N(C)C(=O)[C@@H](NC(=O)[C@@H](NC)C(C)C)C(C)C]
wherein the method comprises the following steps:
(3-1). dissolving a compound 4 in a tenth organic solvent to form a solution J;
(3-2). adding a sufficient amount of diethylamine to the solution J for insulation reaction, removing a Fmoc protecting group;
(3-3). after the reaction finishes, adding an appropriate amount of eleventh organic solvent and purified water to the reaction system of step (3-2) for extraction, collecting an organic phase, drying by anhydrous sodium sulfate and concentrating under reduced pressure;
(3-4). carrying out chromatographic purification on the concentrate under reduced pressure obtained in step (3-3) by an elution system of toluene:methanol, and concentrating the collected eluent under reduced pressure;
(3-5). after dissolving the concentrated product under reduced pressure obtained in step (3-4) with a twelfth organic solvent, filtering, and concentrating the filtrate under reduced pressure; and
(3-6). vacuum-drying the concentrated product under reduced pressure obtained in step (3-5) to obtain the MMAE.

62. The method according to claim 61, wherein the tenth organic solvent in step (3-1) is selected from dichloromethane, acetonitrile, trichloromethane and carbon tetrachloride; and preferably, the tenth organic solvent in step (3-1) is dichloromethane.

63. The method according to claim 6L wherein the weight-to-volume ratio (g/mL) of the compound 4 to the tenth organic solvent in step (3-1) is about 1:7; preferably, the weight-to-volume ratio (g/mL) of the compound 4 to the tenth organic solvent in step (34) is 1:4-10; and more preferably, the weight-to-volume ratio (g/mL) of the compound 4 to the tenth organic solvent in step (3-1) is 1:5-8.

64. The method according to claim 61, wherein the weight-to-volume ratio (g/mL) of the compound 4 in step (3-1) to the diethylamine in step (3-2) is about 1:3.5; preferably, the weight-to-volume ratio (g/mL) of the compound 4 in step (3-1) to the diethylamine in step (3-2) is 1:3-5; and more preferably, the weight-to-volume ratio (g/mL) of the compound 4 in step (3-1) to the diethylamine in step (3-2) is 1:3-4.

65. The method according to claim 61, wherein the diethylamine in step (3-2) is added dropwise, and in the dropwise addition process, the internal temperature is kept between 0° C. and and the temperature of the insulation reaction in step (3-2) is 20-30° C.

66. The method according to claim 61, wherein the eleventh organic solvent in step (3-3) is selected from dichloromethane, trichloromethane, carbon tetrachloride and toluene; and preferably, the eleventh organic solvent in step (3-3) is dichloromethane.

67. The method according to claim 61, wherein the weight-to-volume ratio (g/mL) of the compound 4 in step (3-1) to the eleventh organic solvent and purified water in step (3-3) is about 1:7:10; preferably, the weight-to-volume ratio (g/mL) of the compound 4 in step (3-1) to the eleventh organic solvent and purified water in step (3-3) is 1:5-10:5-15; and more preferably, the weight-to-volume ratio (g/mL) of the compound 4 in step (3-1) to the eleventh organic solvent and purified water in step (3-3) is 1:6-8:9-12.

68. The method according to claim 61, wherein the silica gel used in the chromatographic purification in step (3-4) is 200-300 mesh silica gel; the elution system is toluene:methanol in a volume ratio (V/V) of 10-20:1; preferably, the elution system is firstly toluene:methanol in a volume ratio (V/V) of about 20:1; and when TLC detects that only the product is visible, die elution system is changed to toluene:methanol in a volume ratio (V/V) of about 10:1.

69. The method according to claim 61, wherein the developing agent of the TLC detection is toluene:methanol in a volume ratio (V/V) of about 5:1.

70. The method according to claim 61, wherein the twelfth organic solvent in step (3-5) is selected from methanol, toluene and acetonitrile; and preferably, the twelfth organic solvent in step (3-5) is methanol.

71. The method according to claim 61, wherein the weight-to-volume ratio (g/mL) of the compound 4 in step (3-1) to the twelfth organic solvent in step (3-5) is about 1:3-10.

72. The method according to claim 61, wherein the process of the step (3-5) can be repeated 1-5 times.
